(19) United States
(12) Patent Application Publication
(10) Pub. No.: US 2010/0247557 A1
(43) Pub. Date: Sep. 30, 2010

(54) Immunostimulant Composition Comprising At Least One Toll-Like Receptor 7 Or Toll-Like Receptor 8 Agonist And A Toll-Like Receptor 4 Agonist

(75) Inventors: Nicolas Burdin, Ecully (FR); Jean Haensler, Grezieu la Varenne (FR)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SANOFI PASTEUR, Lyon cedex 07 (FR)

(21) Appl. No.: 12/797,107

(22) Filed: Jun. 9, 2010

Related U.S. Application Data
(63) Continuation of application No. 10/596,432, filed on Jun. 13, 2006.

(30) Foreign Application Priority Data
Dec. 19, 2003 (FR) 0314995

(51) Int. Cl. A61K 39/00

(52) U.S. Cl. 424/184.1

(57) ABSTRACT

The invention relates to an immunostimulant composition comprising at least one Toll-like receptor 7 or Toll-like receptor 8 agonist and a receptor 4 agonist. The inventive composition can also comprise a vaccine antigen.

The invention relates to the field of immunostimulant compositions comprising at least one agonist of the Toll-like 7 receptor or of the Toll-like 8 receptor which are present on antigen-presenting cells. More particularly, the invention relates to compositions which additionally comprise an agonist of the Toll-like 4 receptor, and in particular such compositions which additionally comprise a vaccine antigen.

It is known in the prior art to want to increase or orient the immune response induced by the antigens present in a vaccine by means of adjuvants which are chosen from the category of immunostimulants. This may be desirable because the antigen, when administered alone, is not sufficiently immunogenic because in particular of its very high degree of purity, or because it is desired to reduce the quantity of antigens present in the vaccine or the number of boosters to be made, or else because it is desired to extend the period of protection conferred by the vaccine. Sometimes, the aim is to modify qualitatively, rather than quantitatively, the induced response.

Numerous molecules have already been described in relation to their adjuvant properties; however, the main adjuvants currently marketed in vaccines are adjuvants based on aluminum or emulsions.

Thus, among the known prior art, there may be mentioned in particular patent EP636031, which discloses the use of a 1H-imidazo[4,5c]quinoline-4-amine as vaccine adjuvant toward a glycoprotein of the Herpes Simplex 2 virus in guinea pigs. In this document, the administered vaccine does not make it possible to completely prevent the development of the disease during a challenge of the animals with the HSV2 virus, but it makes it possible to reduce the lesions, the vaginal excretion of the virus and the phenomenon of recurrence of the disease.

According to the publication entitled “Adjuvant activities of Immune Response Modifier R848: Comparison with CpG ODN”, by Vasilakos et al., in Cellular Immunology 204, 64-74 (2000), the imidazoquinoline derivative R-848 is described as being an adjuvant of the TH1 type, in a test using, as antigen, ovalbumin administered to mice.

This publication also describes another type of vaccine adjuvant consisting of oligonucleotides comprising a dinucleotide CG, in which the cytosine is not methylated, In another prior art document consisting of the publication entitled “Human TLR7 or TLR8 independently confer responsiveness to the antiviral compound R-848” by Jurk et al., in Nature Immunology, June 2002, volume 3 No. 6, p 499, it is stated that the Toll-like receptors play an important role in the immune responses to pathogens, the Toll-like 9 receptor being activated by bacterial DNA having nonmethylated CpG units, whereas R-848 activates the cells via the Toll-like 7 receptor and the Toll-like 8 receptor.

In the publication entitled “Novel synthetic LPS receptor agonists boost systemic and mucosal antibody responses in mice”, by Przetak et al., in Vaccine 21 (2003) pages 961-970, chemical compounds having fatty acid chains are described, which compounds lack sugar rings but which have an adjuvant activity toward antigens formed by the tetanus toxin or ovalbumin. These compounds are known to activate a mechanism of action linked to the Toll-like 4 receptor.

All of these compounds are known individually to have immunostimulant properties in various degrees according to the conditions for administration; however, it remains desirable to be able to have a composition which makes it possible to potentiate these immunostimulant properties, in particular in the case of the administration of a vaccine antigen.

To achieve this objective, the subject of the present invention is an immunostimulant composition comprising at least one agonist of the Toll-like 7 receptor or of the Toll-like 8 receptor, which additionally comprises an agonist of the Toll-like 4 receptor. Accordingly, potentiation of the immunostimulant response is obtained.

According to a particular embodiment of the invention, the agonist of the Toll-like 7 receptor or of the Toll-like 8 receptor is a compound different from the agonist of the Toll-like 4 receptor.

According to a particular embodiment, the immunostimulant composition additionally comprises at least one vaccine antigen. Accordingly, the induced immune response against the antigen is potentiated.

According to a particular embodiment of the invention, the agonist of the Toll-like 7 receptor or of the Toll-like 8 receptor is an imidazoquinolineamine derivative. Such an agonist may be obtained by pure chemical synthesis and therefore has all the guarantees of reproducibility and safety necessary for pharmaceutical use.

According to a particular embodiment, the imidazoquinolineamine derivative is 4-amino-2-ethoxymethyl-α,α-dimethyl-1-H-imidazo[4,5c]quinoline-1-ethanol.

According to one embodiment, the agonist of the Toll-like 4 receptor is a compound described in application WO0044758, and in particular ER804057; such a compound, obtained by pure chemical synthesis, also has all the guarantees of reproducibility and safety necessary for pharmaceutical use.

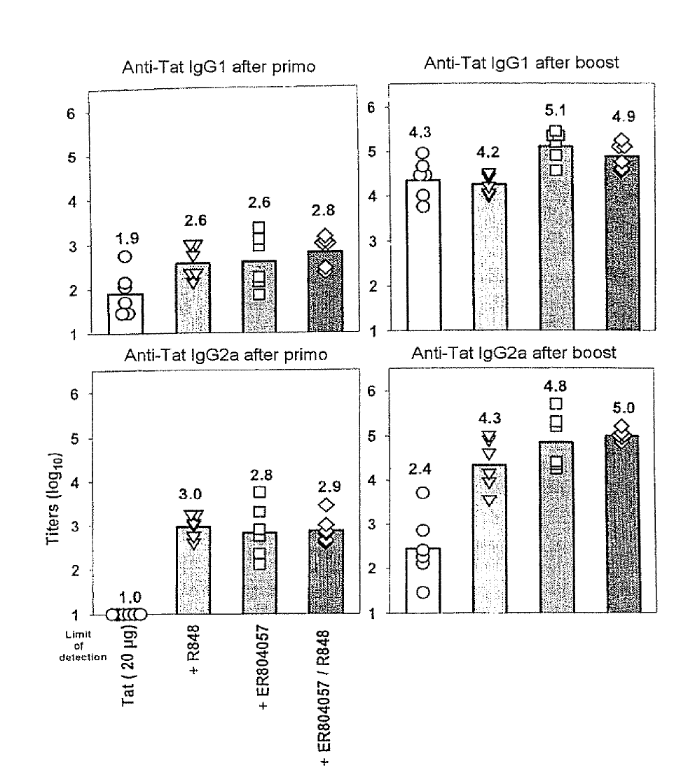

Numerous other advantages of the present invention will emerge in the light of the detailed description which follows, with reference to FIGS. 1 to 4 which illustrate the results obtained in example 5.

The present invention relates to an immunostimulant composition; the expression immunostimulant composition is understood to mean a composition capable of inducing the maturation or the activation of cells of the immune system, such as dendritic cells, which then leads to the expression, on the cells, of certain markers (CD25, CD80, CD83 and the like) which can be detected, or to the secretion of cytokines (IL6, IL12p70, TNF-α, and the like) which can be assayed.

According to a particular embodiment, the immunostimulant composition of the invention comprises at least one vaccine antigen. The expression vaccine antigen is understood to mean an antigen capable of inducing an immune system response when it is administered to humans or to an animal. This immune system response can be manifested by a production of antibodies or by an activation of certain cells, in particular antigen-presenting cells (e.g.: dendritic cells), T lymphocytes and B lymphocytes.

The vaccine composition may be a composition for prophylactic use or for therapeutic use, or both.

It may be administered by any of the routes normally used or recommended for vaccines: parenteral route, mucosal route, and may be provided in various forms: injectable or pulverizable liquid, freeze-dried or spray-dried or air-dried formulation, and the like. It may be administered by means of a syringe or by means of a needle-free injector for intramuscular, subcutaneous or intradermal injection. It may also be administered by means of a nebulizer capable of delivering a dry powder or a liquid spray at the level of the mucous membranes, whether they are nasal, pulmonary, vaginal or rectal.

The vaccine antigens used in the vaccine compositions according to the present invention arc “direct” antigens, that is to say that this is not DNA encoding these antigens, but the antigens themselves; this may be a whole microbe or only a portion of this microbe; accordingly, among the antigens normally used in vaccines, there may be mentioned in particular:
polysaccharides, whether they are alone or conjugated with carrier elements, such as carrier proteins,
attenuated live whole microbes,
inactivated microbes,
recombinant peptides and proteins,
glycoproteins, glycolipids, lipopeptides,
synthetic peptides,
burst microbes in the case of vaccines called “split” vaccines.

These antigens are antigens which are used or are capable of being used for the treatment or prevention of various diseases such as, for example: diphtheria, tetanus, polio, rabies, whooping cough, hepatitis A, B, C, yellow fever, typhoid fever, chicken pox, measles, mumps, rubella, Japanese encephalitis, meningitis, pneumococcal infections, rotavirus infections, AIDS, cancers, tuberculosis, Lyrne's disease, RSV infections, herpes, bacterial conditions caused by Chlamydia, Neisseria gonorrhea; Streptococcus pneumonia; Moraxella catarrhalis, or Haemophilus influenza type B, malaria, leishmaniasis, listeriosis, and the like.

The vaccine composition according to the invention may be a composition intended for immunization against a single pathogen or cancer, that is to say that it comprises one or more antigens from a single pathogen or cancer, or may be a composition intended for immunization against several pathogens or cancers (reference is then made to a vaccine combination).

For the purposes of the present invention, the expression agonist of the Toll-like 7 and Toll-like 8 receptors is understood to mean a compound capable of binding to either of these receptors or to both and of triggering the signaling cascade associated with these receptors, in particular a compound capable of activating the translocation of NF-κB in cells transfected with cDNA encoding either of these receptors, or both.

Among the agonists suitable for the purposes of the invention, there may be mentioned in particular substituted imidazoquinolineamines, and in particular those described in U.S. Pat. No. 5,389,640. Particularly good results were obtained with 4-amino-2-ethoxy-methyl-α,α-dimethyl-1-H-imidazo[4,5c]quinoline-1-ethanol, also called R-848, of which a method of preparation is indicated in examples 99 and 101 of U.S. Pat. No. 5,389,640.

For the purposes of the present invention, the expression agonist of the Toll-like 4 receptor is understood to mean a compound capable of binding to this receptor and of triggering the associated signaling cascade, in particular a compound capable of activating the translocation of NF-κB in cells transfected with cDNA encoding this receptor.

Among the agonists suitable for the purposes of the invention, there may be mentioned the LPSs of Gram-negative bacteria, or, more appropriately, monophosphorylated derivatives of lipids A of these LPSs, and in particular 3D-MPL or monophosphorylated lipid A deacylated at the 3-position which is described by RIBI in UK patent No. 2211502 and in U.S. Pat. No. 4,436,727 and its reissue 4912094. Synthetic analogues of these products such as those described in CORDCA in application WO98/50399, and in particular RC-529, or alternatively those described in application WO02/12258 are also suitable. Likewise, the compounds which are the subject of applications WO95/14026, WO00/00462, WO01/46126 and WO01/46127 in the name of OM Pharma may be suitable.

Preferably, purely synthetic products, free of saccharide ring, such as those described in U.S. Pat. No. 6,290,973 in the name of EISAI CO, and in particular the product called ER 112066, or more preferably still the product called ER804057, are used. This product is a disodium salt of (1R,6R,22R,27R)-1,27-diheptyl-1,27-bisdodecanoyl-9,19-dihydroxy-9,19-dioxido-14-oxo-6,22-bis[(1,3-dioxotetradecyl)amino]-4,8,10,18,20,24-hexaoxa-13,15-diaza-9,19-diphosphoheptacosan which may be obtained according to the method of preparation indicated in patent application WO0044758, for compound No. 50, i.e. the method described on page 32, provided that myristoyl chloride is replaced beforehand with β-ketomyristic acid in the presence of EDC (that is to say 1-(3-dimethylaminopropyl)-3-ethylcarbodiimide hydrochloride) in the step leading from the product 39 to the product 41 described on page 28 of the patent application.

Each of these agonists, whether the agonist of the Toll-like 4 receptor or the agonist of the Toll-like 7 and 8 receptors, is known to have immunostimulant properties.

The importance of the present invention is that the response observed in the context of the present invention is a potentiated response. While the obtaining of such a potentiation could, for a person unfamiliar with the field of immunology, initially appear as being obvious, it should on the contrary be considered as surprising in the particular field of the invention; indeed, experiments have shown that when 2 or more immunostimulants are present in the same composition, it is frequent for the effect produced by one of them to be inhibitory toward the other immunostimulant(s) present, or at least when a product exerts an immunostimulant effect, it is difficult to further increase the response already obtained.

In immunostimulation tests in vitro, there is observed a synergy of the effects of the immunostimulants brought into contact with the cells of the immune system which induce a level of activation of the cells which is quite exceptional, which could not be anticipated from the level of activation induced by each of the immunostimulants used separately.

Likewise, the synergy observed in the response obtained when the immunostimulant composition comprises vaccine antigens, in particular as regards the number of responding subjects with a very good level of response, is quite exceptional, and could not at all be deduced from the responses obtained with each of the adjuvants taken in isolation.

The results obtained using agonists of these Toll-like 7, 8 and 4 receptors are all the more surprising since tests carried out by combining several agonists of other receptors have not led to any potentiation of the effects observed by each of the agonists used in isolation.

The agonists of the Toll-like 4, 7 and 8 receptors of the present invention have the property of adjuvanting the vaccine antigens with which they are administered, which means in general that they are capable of increasing or modifying the immune system response of the organism to which the vaccine composition is administered, compared with the response which would be obtained in their absence. In particular, this may involve an increase in the humoral response, or in the cellular response, or both. The action may also be not an increase in the response, but a different orientation of the induced response: for example, orientation toward a cellular response rather than a humoral response, production of certain cytokines rather than others, production of certain types or subtypes of antibody rather than others, stimulation of certain cells rather than others, and the like. The action of an adjuvant may also consist in increasing the duration of the immune response over time. This may also involve allowing the reduction in the number of administrations necessary to obtain protection of the individual immunized, or the reduction in the quantity of antigens which is contained in the dose administered.

In the case of the present invention, the synergy observed manifests itself essentially by a decrease in the dispersion of the results obtained, in particular as regards the Th1 response.

The adjuvant action of the agonists according to the invention may be obtained either when they are combined with the antigen or with the antigens of the vaccine composition during their administration, i.e. when they are present directly in the vaccine composition, or when they are administered separately from the antigen or antigens for which it is desired to modify the immunogenicity. It is however preferable to use them in the same vaccine composition as the antigen or the antigens to be administered.

The examples which follow illustrate particular embodiments of the present invention.

1. Preparation of a Stock Suspension of Agonists of the Toll-Like 7 and 8 Receptors. There are available dipalmitoylphosphatidylcholine (DPPC) obtained from Avanti Polar Lipids (Alabaster, Ala.), and 4-amino-2-ethoxymethyl-α,α-dimethyl-1-H-imidazo[4,5c]-quinoline-1-ethanol (R-848) provided by the company InVivogen.

These compounds are provided in powdered form.

342 μg of DPPC (0.46 μmol), supplemented with 150 μg of R-848 (0.51 μmol), are dissolved in 984 μl of a chloroform/methanol 4:1 (vol/vol) mixture. The solution is dried in a round-bottomed glass flask with the aid of a rotary evaporator so as to leave a homogeneous lipid film on the walls of the round-bottomed flask. This film is further dried under a high vacuum in order to remove any trace of residual solvent, and then taken up in 3 ml of water at 60° C. The resulting liposomal suspension is homogenized by vortexing, sonication in an ultrasound bath and then sequentially extruded with the aid of a Lipex extruder thermostated at 50° C., in a passage across a polycarbonate membrane having a porosity of 0.8 μM, followed by a passage across a membrane having a porosity of 0.4 μm and finally a passage across a membrane having a porosity of 0.2 μm.

DPPC/R-848 (0.9:1 mol/mol) liposomes are thus obtained in water at 114 μg/ml of DPPC and 50 μg/ml of R-848.

### 2. Preparation of a Stock Suspension of Agonists of the Toll-Like 4 Receptor.

There are available dipalmitoylphosphatidylcholine (DPPC) obtained from Avanti Polar Lipids (Alabaster, Ala.) and ER804057 provided by the company Eisai.

These compounds are provided in powdered form.

273 μg of DPPC (0.37 μmol), supplemented with 150 μg of ER804057 (0.092 μmol), are dissolved in 760 μl of a chloroform/methanol 4:1 (vol/vol) mixture. The solution is dried in a round-bottomed glass flask with the aid of a rotary evaporator so as to leave a homogeneous lipid film on the walls of the round-bottomed flask. This film is further dried under a high vacuum in order to remove any trace of residual solvent, and then taken up in 3 ml of water at 60° C. The resulting liposomal suspension is homogenized by vortexing, sonication in an ultrasound bath and then sequentially extruded with the aid of a Lipex extruder thermostated at 50° C., in a passage across a polycarbonate membrane having a porosity of 0.8 μm, followed by a passage across a membrane having a porosity of 0.4 μm and finally a passage across a membrane having a porosity of 0.2 μm.

DPPC/ER804057 (4:1 mol/mol) liposomes are thus obtained in water at 91 μg/ml of DPPC and 50 μg/ml of ER804057.

### 3. Preparation of a Stock Suspension of Agonists of the Toll-Like 4 Receptor and Agonists of the Toll-Like 7 and 8 Receptors.

There are available dipalmitoylphosphatidylcholine (DPPC) obtained from Avanti Polar Lipids (Alabaster, Ala.), and 4-amino-2-ethoxymethyl-α,α-dimethyl-1-H-imidazo[4,5c]-quinoline-1-ethanol (R-848) provided by the company InVivogen, and ER804057 provided by the company Eisai.

These compounds are provided in powdered form.

273 μg of DPPC (0.37 μmol), supplemented with 150 μg of TLA4 (0.092 μmol) and with 150 μg of R848 (0.51 μmol), are dissolved in 1.06 ml of a chloroform/methanol 4:1 (vol/vol) mixture. The solution is dried in a round-bottomed glass flask with the aid of a rotary evaporator so as to leave a homogeneous lipid film on the walls of the round-bottomed flask. This film is further dried under a high vacuum in order to remove any trace of residual solvent, and then taken up in 3 ml of water at 60° C. The resulting liposomal suspension is homogenized by vortexing, sonication in an ultrasound bath and then sequentially extruded with the aid of a Lipex extruder thermostated at 50° C., in a passage across a polycarbonate membrane having a porosity of 0.8 μm, followed by a passage across a membrane having a porosity of 0.4 μm and finally a passage across a membrane having a porosity of 0.2 μm.

DPPC/ER804057/R-848 (4:1:5.5 mol/mol/mol) liposomes are thus obtained in water at 91 μg/ml of DPPC, 50 μg/ml of ER804057 and 50 μg/ml of R-848

### 4. Preparation of the Vaccine Compositions

Vaccine compositions are prepared which comprise, as vaccine antigen, a recombinant protein capable of being used in a vaccine against AIDS; it is the detoxified TAT III B protein which is obtained by expression in E. coli and purification by various chromatographic steps, followed by chemical inactivation, as is described in patent application WO99/33346, where it is identified under the term carboxymethylated Tat.

The compositions are prepared in the manner described below.

The liposomal suspensions prepared according to examples 1 to 3 are mixed volume for volume (0.9 ml+0.9 ml) with a concentrated Tat solution at 200 μg/ml in 100 mM Tris buffer containing 200 mM NaCl, pH 7.4, in order to obtain the preparations (1.8 ml final) whose composition is indicated below and in which the quantities of antigens and of adjuvant are indicated per 200 μl dose.

### 1) Tat (20 μg)

2) Tat (20 μg)+ER804057/DPPC (5 μg/9.1 μg, that is 3.1 nmol/12.4 nmol)
3) Tat (20 μg)+ER804057/DPPC/R-848 (5 μg/9.1 μg/5 μg, that is 3.1 nmol/12.4 nmol/16.7 nmol)
4) Tat (20 μg)+R-848/DPPC (5 μg/11.4 μg, that is 16.7 nmol/15.5 nmol).

### 5. Immunization Test on Mice.

There are available 4 groups of 6 female BALB/c mice 8 weeks old to which one of the compositions prepared in example 4 is injected subcutaneously at the rate of a dose of 200 μl per mouse; the injections are performed on D0 and at D21.

Blood samples are collected at the retro-orbital sinus at D14 for assessing the primary response and at D32 for the secondary response. The determination of the level of specific IgG1 and IgG2a is carried out by virtue of the standard ELISA tests.

The mice are sacrificed at D37; their spleen is removed and the splenocytes are isolated.

The results obtained as regards the humoral responses are summarized in the table below and in FIGS. 1 to 4, where the IgG levels are expressed as arbitrary ELISA units (log 10).

For each group of mice, the value indicated in the table is the mean geometric titer of the values obtained for each of the mice.

| Vaccine | IgG1 | IgG2a | IgG1 | IgG2a | IgG1/IgG2a |
|---|---|---|---|---|---|
| composition | at D14 | at D14 | at D32 | at D32 | ratio at D32 |
| Tat | 1.897 | 1.000 | 4.343 | 2.436 | 176.2 |
| Tat + ER804057 | 2.598 | 2.820 | 5.101 | 4.838 | 3.5 |
| Tat + R848 | 2.568 | 2.959 | 4.248 | 4.328 | 1.3 |
| Tat + ER804057 + | 2.805 | 2.864 | 4.877 | 4.989 | 0.9 |
| R848 | | | | | |

The IgG1/IgG2a ratio makes it possible to assess the orientation of the immune response induced. Indeed, a Th1 type response is manifested in mice by a higher proportion of IgG2a, whereas a Th2 type response is manifested by a higher proportion of IgG1. It can therefore be seen that, by virtue of the composition according to the invention, the response is oriented toward the Th1 type a lot more strongly than if each of the immunostimulants were used individually.

The graphs represented in FIGS. 1 to 4 make it possible to visualize the responses obtained for each of the mice, and therefore to assess the greater or lesser dispersion of the results. The performance of the composition according to the invention is particularly notable at the level of the IgG2a response obtained after the injection of the booster; indeed, while the response levels obtained with the compositions having a single immunostimulant, whether R-848 or ER804057, are on average satisfactory, it is noted that the results are in these cases relatively dispersed; whereas with the composition according to the invention all the mice produced a high IgG2a level. This performance is very important in the field of vaccination where it is always desired to protect all the vaccinated subjects, but where the variabilities generally observed between the individuals do not make it possible to ensure the same benefit to each of the individuals receiving the vaccine.

These results, which are observed by presenting in the same vaccine composition an adjuvant comprising both an agonist of the Toll-like 4 receptor and an agonist of the Toll-like 7 and Toll-like 8 receptors, are all the more surprising since tests carried out by combining an agonist of the Toll-like 7 and Toll-like 8 receptors and an agonist of another receptor also present on antigen-presenting cells, have not made it possible to improve the responses compared with the responses obtained using, as adjuvant, each of the compounds separately.

To assess the effect of the pharmaceutical compositions according to the invention on the cellular response, counts are carried out of spleen cells capable of producing γ-interferon by an ELISPOT test. This test is carried out both on fresh cells and on restimulated cells.

To carry out the test, the spleen cells are cultured in cell culture plates at the rate of 200 000 cells per well, in the presence either of the medium alone, or of the recombinant TAT antigen. After 16 hours of culture, the ELISPOT is visualized, i.e. the number of spots corresponding to the cells secreting γ-interferon is counted. The results obtained are summarized in the tables below; the values indicated are the mean values (per group of mice), of the differences calculated for each mouse between the number of spots counted per million of cells in the wells having the recombinant TAT and the number of spots counted per million of cells in the wells having only the medium.

The table below summarizes the results obtained on fresh cells.

| Immunostimulant composition tested | Number of spots per million cells |
|---|---|
| TAT at 20 μg | 7 |
| TAT at 20 μg + R-848 | 25 |
| TAT at 20 μg + ER804057 | 53 |
| TAT at 20 μg + R-848 + ER804057 | 110 |

The table below summarizes the results obtained on cells restimulated in vitro for 7 days, in the presence of IL2, by an overlapping peptide pool completely covering the sequence of the TAT protein.

| Immunostimulant composition tested | Number of spots per million cells. |
|---|---|
| TAT at 20 μg | 33 |
| TAT at 20 μg + R-848 | 518 |
| TAT at 20 μg + ER804057 | 488 |
| TAT at 20 μg + R-848 + ER804057 | 1005 |

In addition, there is carried out in parallel the measurement, by an ELISA test, of the secretion of the IL5 cytokines and of γ-interferon in culture supernatants comprising splenocytes cultured in the presence or otherwise of recombinant TAT for 5 days The results obtained, expressed in μg/ml, are summarized in the table below:

| Immunostimulant composition tested | IL-5 | INF-γ |
|---|---|---|
| TAT at 20 μg | 2893 | 7726 |
| TAT at 20 μg + R-848 | 152 | 8326 |
| TAT at 20 μg + ER804057 | 220 | 3886 |
| TAT at 20 μg + R-848 + ER804057 | 167 | 13887 |

These results show the particularly beneficial effect obtained on the TH1 response, by virtue of the compositions according to the present invention

### 6. Preparation of Liposome Suspensions for the Tests of Stimulation of Human Cells.

There are available dipalmitoylphosphatidylcholine (DPPC) obtained from Avanti Polar Lipids (Alabaster, Ala.), and 4-amino-2-ethoxymethyl-α,α-dimethyl-1-H-imidazo[4,5c]-quinoline-1-ethanol (R-848) provided by the company InVivogen.

These compounds are provided in powdered form.

9.92 mg of DPPC (115 μmol), supplemented with 1 mg of R-848 (3.38 μmol), are dissolved in 2 ml of a chloroform/methanol 4:1 (vol/vol) mixture. The solution is dried in a round-bottomed glass flask with the aid of a rotary evaporator so as to leave a homogeneous lipid film on the walls of the round-bottomed flask. This film is further dried under a high vacuum in order to remove any trace of residual solvent, and then taken up in 4 ml of water at 60° C. The resulting liposomal suspension is homogenized by vortexing, sonication in an ultrasound bath and then sequentially extruded with the aid of a Lipex extruder thermostated at 50° C., in a passage across a polycarbonate membrane having a porosity of 0.8 μm, followed by a passage across a membrane having a porosity of 0.4 μm and finally a passage across a membrane having a porosity of 0.2 μm.

DPPC/R-848 (4:1 mol/mol) liposomes are thus obtained in water at 2.48 mg/ml of DPPC and 250 μg/ml of R-848.

There are available dipalmitoylphosphatidylcholine (DPPC) obtained from Avanti Polar Lipids (Alabaster, Ala.) and ER804057 provided by the company Eisai.

These compounds are provided in powdered form.

19 mg of DPPC (25 μmol), supplemented with 11 mg of ER804057 (6.7 μmol), are dissolved in 5 ml of a chloroform methanol 4:1 (vol/vol) mixture. The solution is dried in a round-bottomed glass flask with the aid of a rotary evaporator so as to leave a homogeneous lipid film on the walls of the round-bottomed flask. This film is further dried under a high vacuum in order to remove any trace of residual solvent, and then taken up in 11 ml of water at 60° C. The resulting liposomal suspension is homogenized by vortexing, sonication in an ultrasound bath and then sequentially extruded with the aid of a Lipex extruder thermostated at 50° C., in a passage across a polycarbonate membrane having a porosity of 0.8 μm, followed by a passage across a membrane having a porosity of 0.4 μm and finally a passage across a membrane having a porosity of 0.2 μm.

DPPC/ER804057 (4:1 mol/mol) liposomes are thus obtained in water at 1.72 mg/ml of DPPC and 1 mg/ml of ER804057.

### 7. Test of Stimulation of Human Cells In Vitro.

The capacity of the compositions according to the invention to induce the maturation of dendritic cells derived from human monocytes in vitro is evaluated for 4 independent donors. The monocytes are obtained from peripheral blood mononuclear cells and are cultured for 5-6 days in the presence of IL4 and of GM-CSF.

These cells are then cultured for 2 days in the presence of one of the following compositions:
culture medium alone, serving as negative control,
R-848/DPPC liposomes prepared according to example 6 and diluted so as to obtain 2.96 μg/ml of R-848,
ER804057/DPPC liposomes prepared according to example 6 in an amount of 0.1 μg/ml,
a combination of the 2 liposomal preparations.

There are then carried out a phenotype analysis by flow cytometry, making it possible to measure the expression of the maturation markers CD25, CD80 and CD83, and an ELISA measurement of the cytokines (TNF-α, IL6 and IL12p70) secreted by these cells.

The results indicated in the tables below represent the mean values calculated for the 4 donors:

| | Percentage of cells | | |
|---|---|---|---|
| | expressing the markers | | |
| | CD25 | CD80 | CD83 |
| Medium alone | 3 | 12 | 4 |
| R-848 | 25 | 34 | 19 |
| ER804057 | 35 | 46 | 15 |
| ER804057 + R-848 | 78 | 60 | 33 |

| | Quantity of cytokines in pg/ml | | |
|---|---|---|---|
| | TNF-α | IL 6 | IL12p70 |
| Medium alone | 61 | 77 | 10 |
| R-848 | 1727 | 8263 | 288 |
| ER804057 | 398 | 8349 | 22 |
| ER804057 + R-848 | 12041 | 69973 | 5304 |

The results obtained show the high capacity of the compositions according to the invention to induce the secretion of cytokines indicating a TH1 oriented response, such as IL12p70; the synergy obtained by combining the 2 products is remarkable. The compositions according to the invention are therefore particularly recommended in all the methods of treatment in which it is sought to obtain a Th1 oriented immune system response, and in particular all the cases where it is desirable to induce the secretion of one of the following cytokines: TNF-α, IL-6 or IL12p70.

1. An immunostimulant composition comprising at least one agonist of the Toll-like 7 receptor or of the Toll-like 8 receptor, wherein the composition additionally comprises an agonist of the Toll-like 4 receptor.

2. The immunostimulant composition as claimed in the preceding claim, wherein the agonist of the Toll-like 7 receptor or of the Toll-like 8 receptor is a compound different from the agonist of the Toll-like 4 receptor.

3. The immunostimulant composition as claimed in claim 1, it wherein the composition additionally comprises at least one vaccine antigen.

4. The immunostimulant composition of claim 1, wherein the agonist of the Toll-like 7 receptor is an imidazoquinolineamine derivative.

5. The immunostimulant composition as claimed in the preceding claim, wherein the imidazoquinolineamine derivative is 4-amino-2-ethoxymethyl-α,α-dimethyl-1-H-imidazo[4,5e]quinoline-1-ethanol.

6. The immunostimulant composition as claimed in claim 1, wherein the agonist of the Toll-like 4 receptor is ER804057.

7.-8. (canceled)

9. The immunostimulant composition as claimed in claim 5, wherein the agonist of the Toll-like 4 receptor is ER804057.
